(19) United States
(12) Patent Application Publication
(10) Pub. No.: US 2006/0025836 A1
(43) Pub. Date: Feb. 2, 2006

(54) Device to alleviate freezing of gait in users with Parkinsonism

(76) Inventors: Jay Arthur Van Gerpen, Atlanta, GA (US); Charles T. Rucker, Alpharetta, GA (US)

Correspondence Address: WOMBLE CARLYLE SANDRIDGE & RICE, PLLC, P.O. BOX 7037, ATLANTA, GA, 30357-0037, US

(21) Appl. No.: 11/121,571

(22) Filed: May 4, 2005

Related U.S. Application Data
(60) Provisional application No. 60/598,174, filed on Aug. 2, 2004.

(51) Int. Cl. A61N 5/06

(52) U.S. Cl. 607/88

(57) ABSTRACT

A device for alleviating freezing of gait in a user with parkinsonism comprising a light source adjustably seated on a holder is provided. A method for alleviating freezing of gait also is provided.

### CROSS-REFERENCE TO RELATED APPLICATION

This application claims priority to U.S. Provisional Application Ser. No. 60/598,174, filed Aug. 2, 2004, which is incorporated by reference herein in its entirety.

### FIELD OF THE INVENTION

The present invention generally relates to a treatment for a sufferer of parkinsonism, and in particular, to an aide or device for providing a constant or recurring stimulus to reduce or eliminate the occurrence of “Freezing of Gait” (FOG) in such a user.

### BACKGROUND OF THE INVENTION

Parkinsonism is a debilitating syndrome, encompassing several neurological diseases that compromise the motor skills of its victims. Such diseases include Parkinson's Disease, Normal Pressure Hydrocephalus (NPH), multiple systems atrophy (MSA), progressive supranuclear palsy (PSP) and others. As parkinsonism progresses, one particularly debilitating problem is “Freezing of Gait” (FOG), in which the user locks up or becomes so fixated they are unable to move or initiate further stepping movements by their own volition. In some cases the user feels “stuck to the floor” as if by a magnetic force. Unfortunately, FOG is not responsive to available medications. As a result, FOG poses a significant risk of injury to users if they are left unattended, even when trying to perform the most mundane tasks, and especially when they are in public settings, such as trying to cross a street. Accordingly, users suffering from FOG require constant monitoring and assistance. Thus, there is a need for a treatment for users suffering from FOG.

### SUMMARY OF THE INVENTION

The present invention is directed generally to a device for alleviating freezing of gait in a user with parkinsonism or other neuro-muscular disease. The device comprises a light source, for example, a laser, adjustably seated in a holder. The light source is focused into a visual cue, for example, a line or other shape. The device may include a clip, strap, band, or other means of detachably affixing the device to the clothing or an accessory of the user. As the user steps toward the visual cue, the visual cue advances with the user, thereby creating a continuing, successive stimulus for eliminating freezing for each step.

The present invention further is directed generally to a method of alleviating freezing of gait in a user with parkinsonism or other neuro-muscular disease. The method includes providing a device including a light source, and forming a visual cue from the light source. The device is positioned relative to the user such that the light source is projected in a direction of movement of the user. As the user moves, the visual cue progressively is advanced with the user to promote step initiation.

Other aspects and features of the present invention will become apparent in view of the figures and the following description.

### BRIEF DESCRIPTION OF THE DRAWINGS

FIG. 1 depicts an exemplary device according to the present invention;

FIG. 2 depicts an exemplary light source that may be used according to the present invention, with a hood;

FIG. 3 depicts an exemplary device according to the present invention, after being angularly adjusted;

FIG. 4 depicts an exemplary device according to the present invention with an optional band;

FIG. 5 depicts an exemplary device according to the present invention with an optional strap for attachment to the head, waist, leg, or accessory of the user;

FIG. 6 depicts an exemplary device according to the present invention removably affixed to an optional head strap;

FIG. 7 depicts an exemplary device according to the present invention in use;

FIG. 8 depicts another exemplary device according to the present invention in use; and

FIG. 9 depicts yet another exemplary device according to the present invention in use.

### DETAILED DESCRIPTION OF THE INVENTION

The present invention generally is directed to a device and method for alleviating “Freezing of Gait” (FOG) in a user suffering from parkinsonism or a similar neurological ailment. The device and method of the present invention provide a visual clue to the user to cause or trigger an autonomic response. In one aspect, the visual clue is a line or other pattern orthogonal to the direction of motion of the user, typically on the floor or ground. It has been found that such a line can trigger a response in parkinsonism users to step on or across the visual cue, thereby eliminating the freezing for at least that step. Successive visual cues are created as the user moves forward. The device and method of the present invention provide a convenient, portable, versatile means of using a visual cue to alleviate FOG.

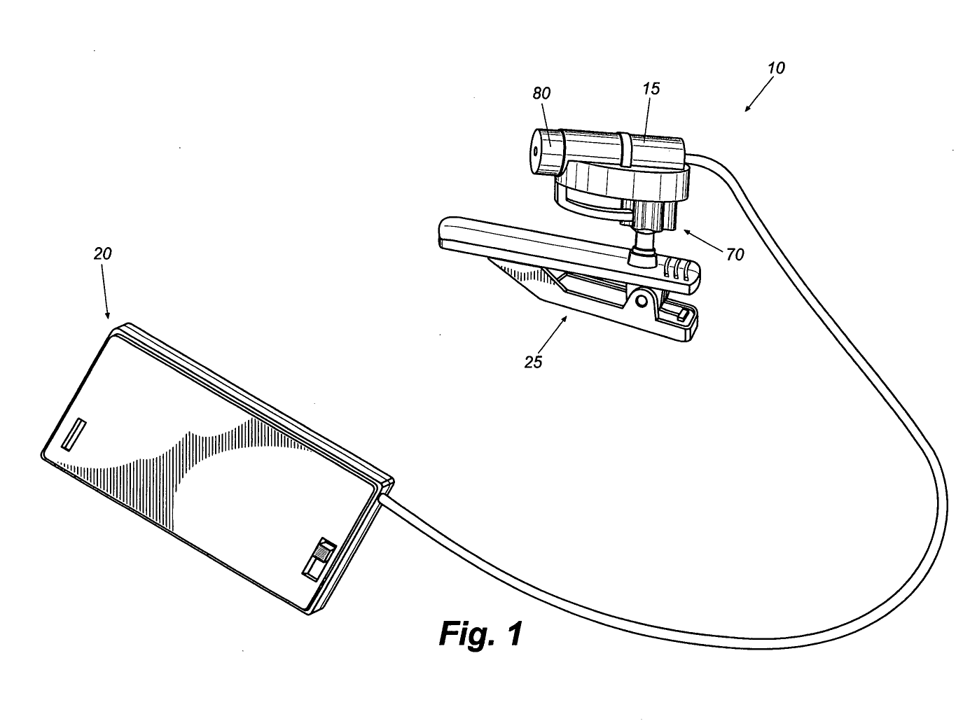

FIG. 1 depicts an exemplary device according to the present invention. The device 10 includes a light source 15, a power source 20, and a holder 25. The light source may be focused using a shape generator 80 into any shape as desired, for example, a solid or dashed line, square, oval, circle, polygon, or any other illuminated shape or pattern. Additionally, the shape or pattern may be adjustable in size, provided that it is sufficiently visible to provide the visual cue or stimulus for the user. Thus, for example, where the shape is a line, the line may have a thickness of from about 0.5 mm to about 3 mm and a length of from about 450 mm to about 750 mm. However, it will be understood that the exact dimensions of the line depend on the fan angle of the laser and the distance between the laser or line generator and the illuminated surface.

The light source generally is selected to have sufficient brilliance and/or power to generate the visual cue at a desired distance in front of the user. Depending on where the device is mounted (as will be discussed in detail below), the light source may have sufficient brilliance and/or power to generate the visual cue at a distance of from about 0.25 m to about 3 m, for example, from about 0.5 m to about 1.5 m.

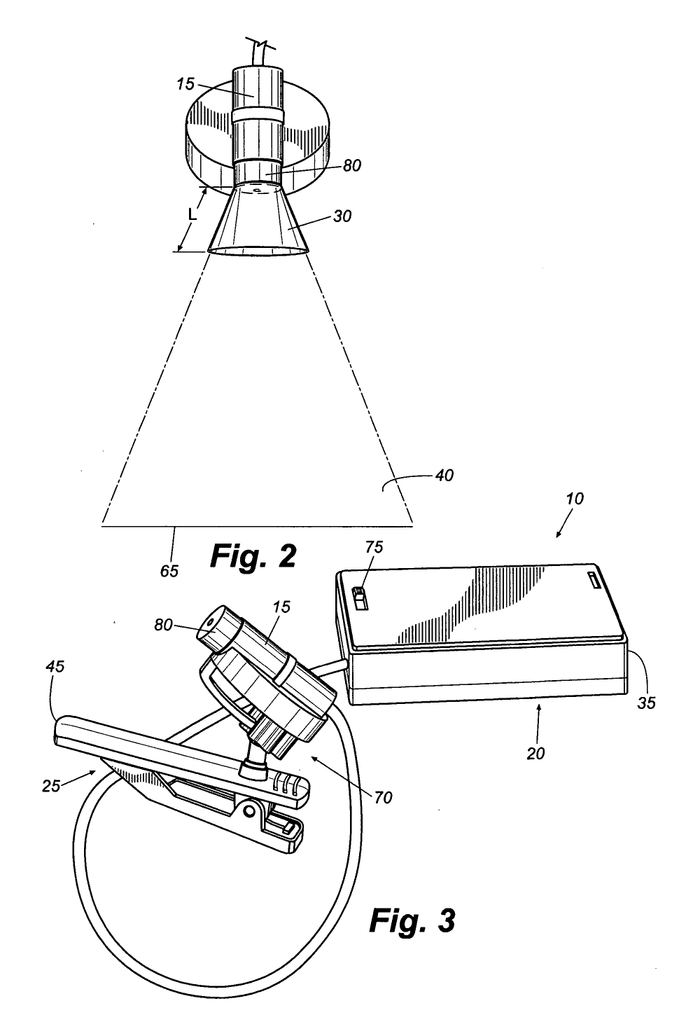

Any suitable light source may be used with the present invention, and in one aspect, the light source is a laser. For example, the laser may be a red laser, such as a red Class IIIA laser having a wavelength of about 635 nm. Alternatively, the laser may be a green laser having a wavelength of about 530 nm. Use of a green laser may be advantageous in some instances, for example, where the device is being used outdoors or in other brightly-lit areas, where red lasers are less perceptible. Optionally, where higher power lasers or other similar light sources are used to enhance visibility of the visual cue, a hood 30 such as that shown in FIG. 2 can be provided to enclose the laser beam 40 and ensure that the laser line 65 cannot be viewed too closely at full power. In one aspect, the hood 30 has a length L of from about 5 to about 150 mm. In another aspect, the hood 30 has a length L of from about 10 to about 100 mm, for example, 20 to about 50 mm, although it will be understood that other dimensions sufficient to substantially prevent close, direct eye contact therewith also can be used. The device 10 may be provided with a feature (not shown) that disables the power source, and thus the laser, if the hood 30 is tampered with or removed. Although lasers are discussed in detail herein, it should be understood that other light sources may be used in accordance with the present invention. For example, the light source may be a focused beam flashlight or pen-light, stroboscopic light, or the like.

The light source may be powered by any suitable energy source, for example, one or more batteries. Any size battery may be used as needed to supply sufficient power to the light source. Additionally, the battery or batteries may be rechargeable. Rechargeable batteries also may be used to stabilize voltage or current of the power source. If desired, a pressure sensitive ceramic, an electromechanical generating device, or a similar medium may be used to produce the necessary power for the light source. The power source 20 may be enclosed in a housing or pack 35, which may include a band, clip, or other fastener to secure the housing to a belt, walker, coat pocket, or the like.

If desired, the power source may be interconnected with an “on/off” switch 75. The user can switch the device to the “on” position so that the visual cue is projected until the user manually switches the device to the “off” position. A light emitting diode (LED) may be provided to indicate whether the device is on. In one aspect, a pulsed LED is used to conserve power and increase battery life. Alternatively, the device may be provided with a timer (not shown) that automatically turns the device off after a certain amount of time. Alternatively still, the device may be provided with a motion or other sensor (not shown) that automatically turns the device off after a period of inactivity. Likewise, the device may be provided with a sensor that automatically turns the device on when motion is detected. In some such aspects, the sensor further may be adapted to detect motion in a desired direction or at a minimum height, such as detecting a forward motion or movement of the user, so that motion caused by shaking of the user would not necessarily activate the device.

As generally illustrated in FIGS. 1 and 3-8, the light source 15 of the device of the present invention may be mounted in, form part of, or be integral with a holder 25, such as a bracket or clip, band, arm band, leg band, or ankle band, lanyard, head strap, belt, helmet, shoe, or other attachment mechanism or holder as desired for mounting the light source on the clothes or person of the wearer or on a walker, cane, chair, or similar assistance device for the user. In one aspect shown in FIG. 3, the light source 15 is mounted adjustably in the holder 25 using a universal or similar swivel joint 70, so that the user can position the light source 15 at a multitude of angles and directions as needed to optimize visibility of the visual cue. For example, some users may prefer the visual cue to be from 0.025 m to about 0.25 m from the user's feet. Other users may prefer the visual cue to be from 0.25 m to about 0.5 m from the user's feet. Still other users may prefer the visual cue to be from 0.5 m to about 0.75 m from the user's feet. Yet other users may prefer the visual cue to be from 0.75 m to about 1 m from the user's feet. Others may require greater distances. Greater or lesser distances also may be preferred, depending on the needs of the individual. The light source further may be adjusted in any direction using a swivel or universal joint, locking type hinge, goose-neck, or any other, similar adjustable support means.

In one aspect of the present invention shown in FIGS. 1 and 3, the holder 25 comprises a clip 45. The clip may be a spring-based clamp or tongue, or any other clip as desired. The user can attach removably the clip to an article of clothing or accessory, such as a shirt, waistband, belt, shoe, or hat. Alternatively, the user can attach removably the clip to an implement, such as a walker, cane, bicycle, shopping cart, vacuum cleaner, lawnmower, and so on. The user then can adjust the angle of the light source using the swivel joint 70 to optimize the position of the visual cue on the floor or ground.

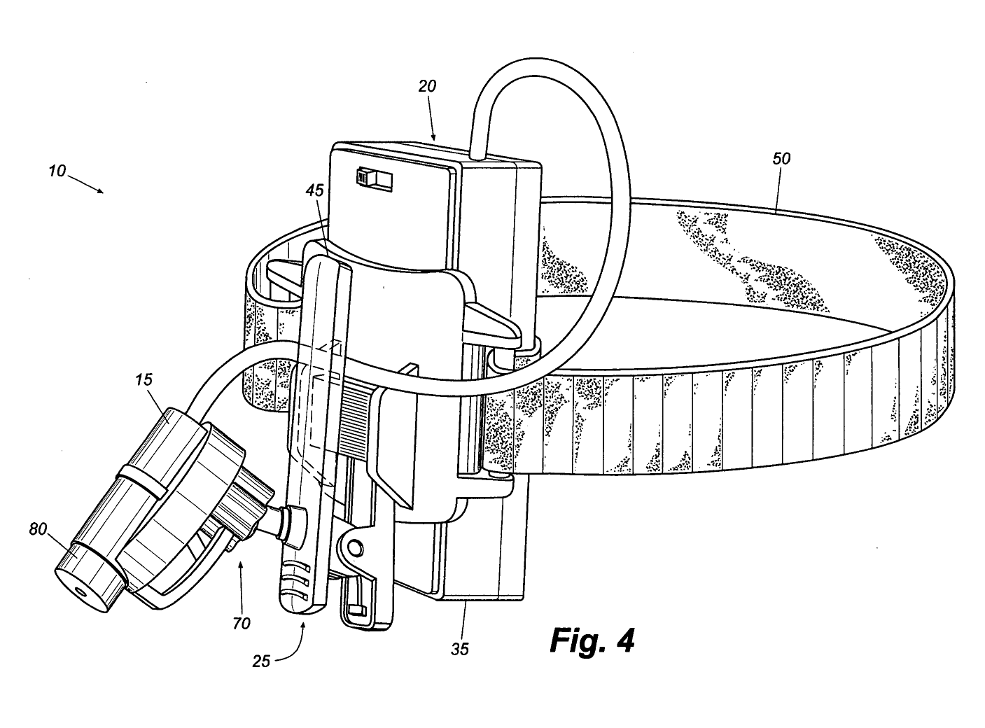

In another aspect shown in FIG. 4, the holder 25 comprises a band 50. The band 50 may be positioned around the head, neck, arm, wrist, leg, waist, ankle, or other part of the user. Alternatively, the user may attach the device to an implement including, but not limited to, those described above. The band also may be adjustable, for example, using a hook and loop fastener (for example, VELCRO®), elastic bands, ties, locking loops, or other fasteners.

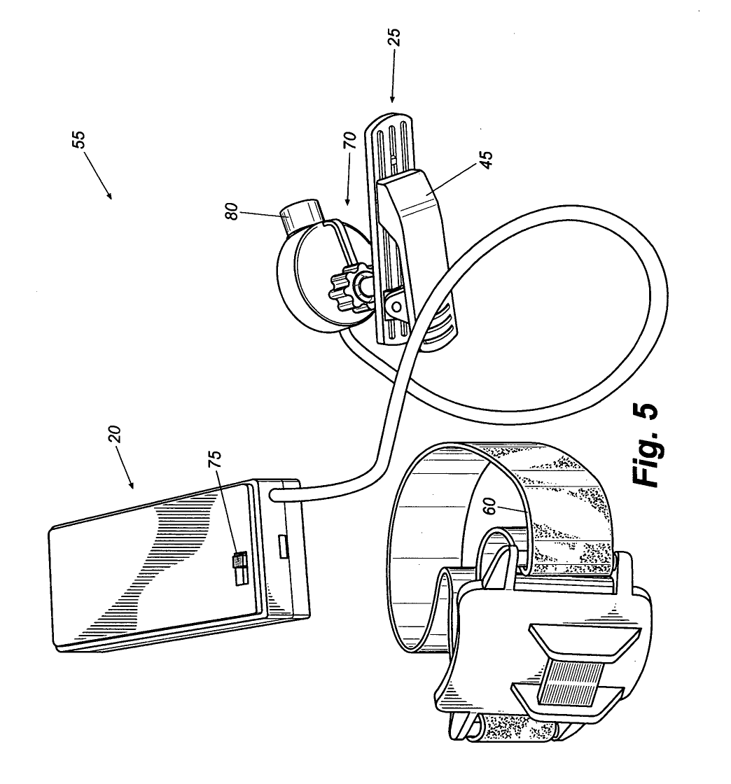

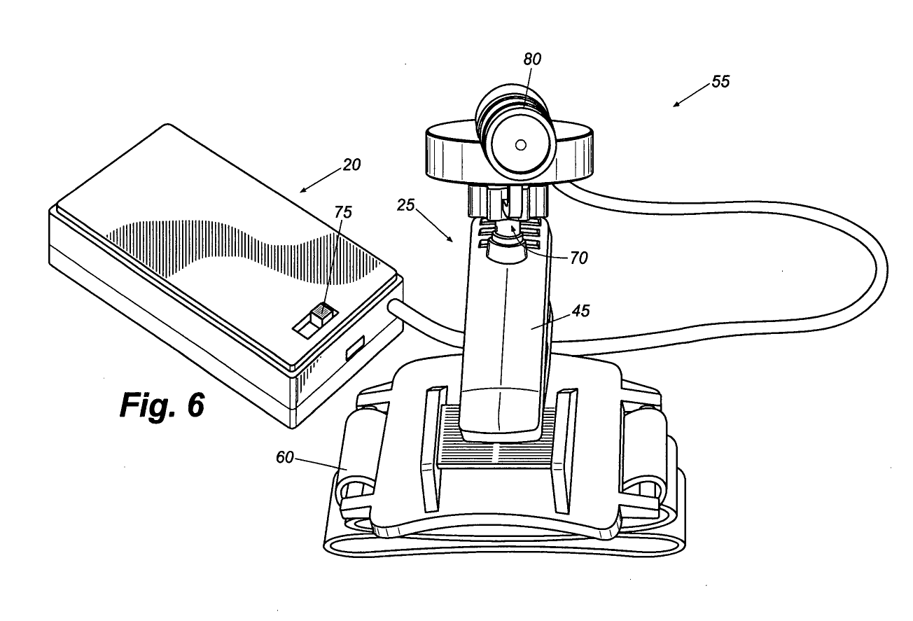

FIGS. 5 and 6 depict another example enhancement of the device 55 according to the present invention in which the holder 25 comprises a head strap 60. The light source 15 may be attached removably to the head strap 60 with or without a clamp, or about or over clip 45 (shown in FIG. 6). The head strap may be worn on the head of the user or on a hat or helmet worn by the user. If desired, the user may be provided with a clip, band, and head strap that may be interchanged and attached to the light source for use. Other types of fasteners also may be provided and are contemplated hereby.

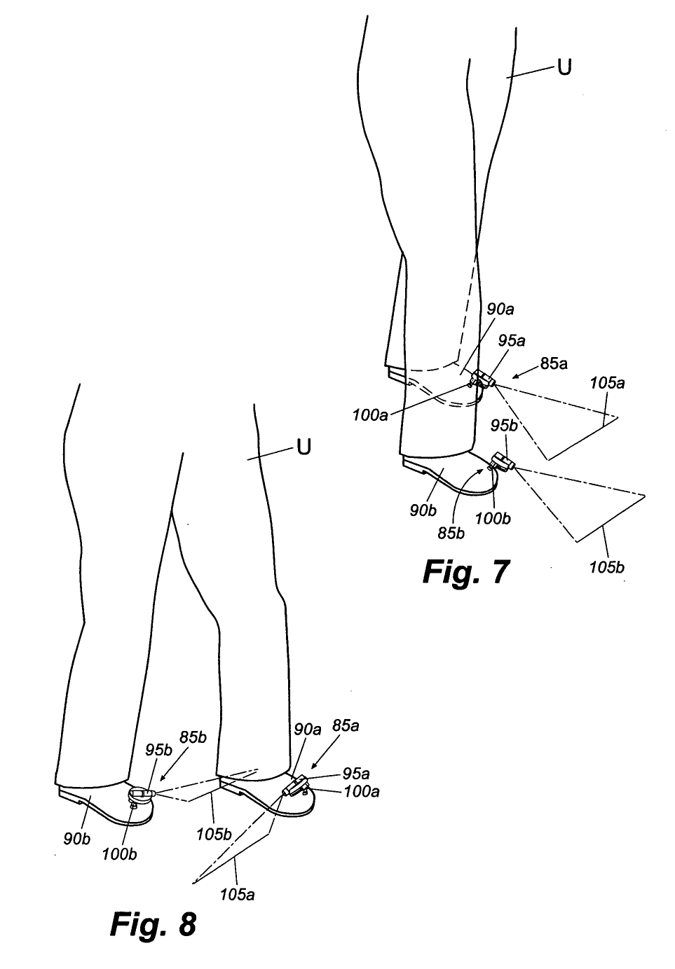

Turning to FIGS. 7 and 8, another example device 85a, 85b is provided as part of a pair of shoes 90. The device 85a, 85b may be mounted on, attached to, or integral with one or both shoes 90a, 90b. The device 85a, 85b includes a light source 95a, 95b that may be adjusted in any direction using a swivel or universal joint 100a, 100b, locking type hinge, goose-neck, or any other, similar adjustable support means. The power source (not shown) for the device 85a, 85b may be housed within the shoe 90a, 90b. In this aspect, the device 85a on the left shoe 90a provides a visual cue 105a to assist the user U with moving the right leg forward. Likewise, the device 85b on the right shoe 90b provides a visual cue 105b to assist the user U with moving the left leg forward. Other features described herein may be incorporated into this aspect as desired.

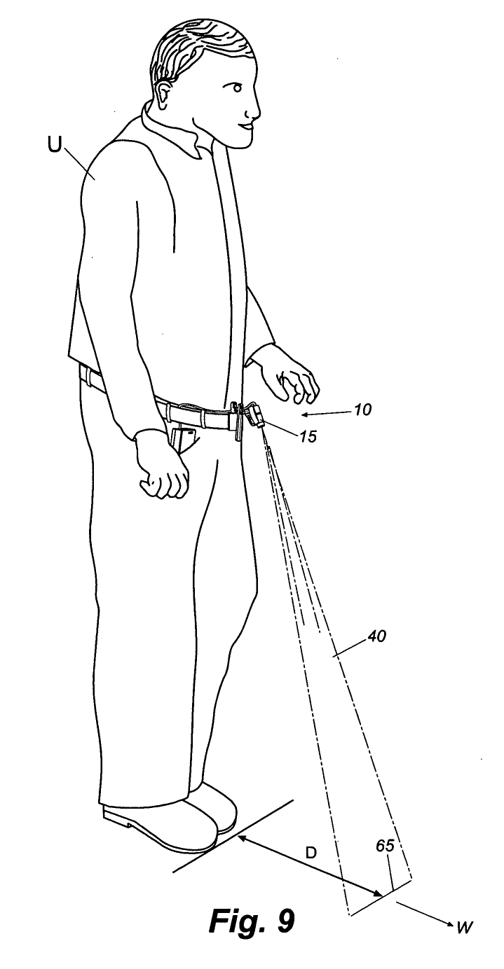

According to yet another aspect of the present invention, a method of alleviating FOG is provided. The method generally comprises providing a light source and focusing the light source into a visual cue. As shown in FIG. 9, the position and/or orientation or altitude of the light source 15 can be adjusted relative to the body and/or posture of a user so that the visual cue 65 is projected in the walking path W of the user U at a distance and location with respect to the user's field of vision so as to be readily viewable or detected by the user. The light source 15 may be clipped or otherwise removably attached to the clothing, person, or an accessory of the user U. The light source 15 typically will be adjusted so as to be projected at a desired or preferred distance D and/or location in front of the user, for example, a distance of from about 0.25 m to about 1 m from the feet of the user to provide a visual cue on which the user can concentrate or focus to continue their forward movement. As the user U steps toward, on, or over the visual cue 65, the visual cue 65 also moves forward. Thus, the visual cue assists the user with moving forward, step by step to aid in continued mobility of the user.

Accordingly, it will be readily understood by those persons skilled in the art that, in view of the above detailed description of the invention, the present invention is susceptible of broad utility and application. Many adaptations of the present invention other than those herein described, as well as many variations, modifications, and equivalent arrangements will be apparent from or reasonably suggested by the present invention and the above detailed description thereof, without departing from the substance or scope of the present invention.

While the present invention is described herein in detail in relation to specific aspects, it is to be understood that this detailed description is only illustrative and exemplary of the present invention and is made merely for purposes of providing a full and enabling disclosure of the present invention. The detailed description set forth herein is not intended nor is to be construed to limit the present invention or otherwise to exclude any such other embodiments, adaptations, variations, modifications, and equivalent arrangements of the present invention, the present invention being limited solely by the claims appended hereto and the equivalents thereof.

What is claimed is:

1. A device for alleviating freezing of gait in a user with parkinsonism comprising a light source adjustably seated in a holder.

2. The device of claim 1, further comprising a releasable clamp attached to said holder.

3. The device of claim 1, wherein said light source projects a line or pattern.

4. The device of claim 1, wherein said light source projects a pattern at a distance of from about 0.25 m to about 3 m.

5. The device of claim 1, wherein said light source continuously projects a pattern.

6. The device of claim 1, wherein said light source intermittently projects a pattern.

7. The device of claim 1, wherein said light source comprises a laser, a focused beam incandescent bulb, or a stroboscopic flash tube.

8. The device of claim 1, wherein said holder comprises a bracket, clip, band, lanyard, strap, belt, hat, helmet, shoe, or any combination thereof.

9. The device of claim 1, further comprising a power source interconnected with said light source, wherein said power source comprises disposable battery, a rechargeable battery, a pressure sensitive medium, an electromechanical generating device, or any combination thereof.

10. The device of claim 1, further comprising a hood surrounding the light source.

11. The device of claim 1, incorporated into a shoe.

12. A device for alleviating freezing of gait in a user comprising:
a light source adapted to project a visual cue at a desired distance in front of the user to trigger a next step by the user; and
a holder for said light source, said holder being mounted detachably on the user or on an appliance of the user, said light source being adjustably oriented at a desired angle with respect to the user to project said visual cue at a desired distance.

13. The device of claim 12, wherein said light source comprises a laser, a focused beam incandescent bulb, or a stroboscopic flash tube.

14. The device of claim 12, wherein said holder comprises a bracket, clip, band, lanyard, strap, belt, hat, helmet, shoe, or any combination thereof.

15. The device of claim 12, further comprising a power source interconnected with said light source.

16. The device of claim 15, further comprising a motion activated sensor interconnected with said power source.

17. The device of claim 15, further comprising a timer interconnected with said power source.

18. A method of alleviating freezing of gait in a user comprising:
providing a device comprising a light source;
forming a visual cue from the light source; and
positioning the device relative to the user such that the light source is projected in a direction of movement of the user;
whereby as the user moves, the visual cue is advanced with the user to promote step initiation.

19. The method of claim 18, further comprising removably affixing the device to the user or to an appliance or accessory of the user.

20. The method of claim 18, further comprising adjusting the light source to position the visual cue at a desired distance.
